(19) United States
(12) Patent Application Publication
(10) Pub. No.: US 2025/0073123 A1
(43) Pub. Date: Mar. 6, 2025

(54) Medical Device Comprising Self-Lubricating Rubber Component

(71) Applicant: Becton, Dickinson and Company, Franklin Lakes, NJ (US)

(72) Inventors: Gumersindo Rodriguez, Sumter, SC (US); Nestor Rodriguez San Juan, Hamburg, NJ (US); Leandro Forciniti, Wharton, NJ (US); Glenn S. Posey, Sumter, SC (US); Alfred Wesley Prais, Hewitt, NJ (US)

(21) Appl. No.: 18/239,896

(22) Filed: Aug. 30, 2023

(51) Int. Cl. A61J 1/05; A61J 1/14; C08K 3/34; C08K 3/36; C08L 9/00

(52) U.S. Cl. CPC A61J 1/05; A61J 1/1468; C08K 3/346; C08K 3/36; C08L 9/00; C08L 2203/02

(57) ABSTRACT

A medical device includes a container defined by walls and having a first end having an opening. The opening is sealed by a rubber component. The rubber component is formed from a self-lubricating rubber formed from a rubber composition including a halogenated isobutylene-isoprene co-polymer and at least one of (1) clay minerals or silica and (2) a liquid polyisoprene and butadiene homopolymer. A rubber component configured for use in a medical device and a rubber composition are also disclosed.

### BACKGROUND OF THE INVENTION

### Field of the Invention

The present disclosure relates generally to medical devices having a self-lubricating rubber component exhibiting low friction and/or low gas/liquid permeability.

### Description of the Related Art

Traditionally, containers for chemically sensitive materials have been made from inorganic materials such as glass. Glass containers offer the advantage that they are substantially impenetrable by atmospheric gases and thus provide a product with a long shelf life. However, glass containers can be fragile and expensive to manufacture.

More recently, lighter and less expensive containers made of polymeric materials are being used in applications in which traditional glass containers were used. These polymeric containers are less susceptible to breakage, lighter, and less expensive to ship than glass containers. However, polymeric containers can be permeable to gases, permitting atmospheric gases to pass through the polymeric container to the packaged product and also permitting gases in the packaged product to escape through the polymeric container, both of which undesirably degrade the quality and shelf life of the packaged product.

Whether the container is formed from glass or polymeric material, reactivity of the interior surface of the container with the contents of the container, such as biological materials and/or drugs, can be problematic. Trace components of the glass or polymeric material may migrate into the container contents, and/or components of the container contents may migrate or react with the interior surface of the container.

Also, certain devices, such as syringe barrels, require slow and controlled initiation and maintenance of sliding movement of one surface over another surface. It is well known that two stationary surfaces having a sliding relationship often exhibit sufficient resistance to initiation of movement that gradually increased force applied to one of the surfaces does not cause movement until a threshold force is reached, at which point a sudden sliding or shearing separation of the surfaces takes place. This sudden separation of stationary surfaces into a sliding relationship is herein referred to as “breakout” or “breakloose”.

“Breakout force” refers to the force required to overcome static friction between surfaces of a syringe assembly that has been previously moved in a sliding relationship, but has been stationary (“parked” or not moved) for a short period of time (for example, milliseconds to hours). A less well known but important frictional force is “breakloose force”, which refers to the force required to overcome static friction between surfaces of a syringe assembly that have not been previously moved in a sliding relationship or have been stationary for longer periods of time, often with chemical or material bonding or deformation of the surfaces due to age, sterilization, temperature cycling, or other processing.

Breakout and breakloose forces are particularly troublesome in liquid dispensing devices, such as syringes, used to deliver small, accurately measured quantities of a liquid by smooth incremental line to line advancement of one surface over a second surface. The problem also is encountered in devices using stopcocks, such as burets, pipets, addition funnels, and the like where careful dropwise control of flow is desired.

The problems of excessive breakout and breakloose forces are related to friction. Friction is generally defined as the resisting force that arises when a surface of one substance slides, or tends to slide, over an adjoining surface of itself or another substance. Between surfaces of solids in contact, there may be two kinds of friction: (1) the resistance opposing the force required to start to move one surface over another, conventionally known as static friction, and (2) the resistance opposing the force required to move one surface over another at a variable, fixed, or predetermined speed, conventionally known as kinetic friction.

The force required to overcome static friction and induce breakout or breakloose is referred to as the “breakout force” or “breakloose force”, respectively, and the force required to maintain steady slide of one surface over another after breakout or breakloose is referred to as the “sustaining force”. Three main factors, sticktion, inertia, and dimensional interference (including morphology) between the two surfaces contribute to static friction and thus to the breakout or breakloose force. The term “stick” or “sticktion” as used herein denotes the tendency of two surfaces in stationary contact to develop a degree of adherence to each other. The term “inertia” is conventionally defined as the indisposition to motion which must be overcome to set a mass in motion. In the context of the present disclosure, inertia is understood to denote that component of the breakout or breakloose force which does not involve adherence.

Breakout or breakloose forces, in particular the degree of sticktion, vary according to the composition and dimensional interference (related to morphology) of the surfaces. In general, materials having elasticity show greater sticktion than non-elastic materials. The length of time that surfaces have been in stationary contact with each other also influences breakout and/or breakloose forces. In the syringe art, the term “parking” denotes storage time, shelf time, or the interval between filling and discharge. Parking time generally increases breakout or breakloose force, particularly if the syringe has been refrigerated or heated during parking.

A conventional approach to overcoming breakout or breakloose has been application of a lubricant to a surface interface. Common lubricants used are silicone or hydrocarbon oils, such as mineral oils, peanut oil, vegetable oils, and the like. Such products have the disadvantage of being soluble in a variety of fluids, such as vehicles commonly used to dispense medicaments. In addition, hydrocarbon oil lubricants are subject to air oxidation resulting in viscosity changes and objectionable color development. Further, they are particularly likely to migrate from the surface to surface interface. Such lubricant migration is generally thought to be responsible for the increase in breakout or breakloose force with time in parking. As a separate issue, the lubricant can also migrate into the contained solution causing undesirable interactions with the active pharmaceutical ingredients or excipients.

Thus, there is a need for a lubricity mechanism to overcome high breakout and breakloose forces whereby smooth transition of two surfaces from stationary contact into sliding contact can be achieved. Also, there is a need for an improved barrier coating to prevent leaching of materials from a container or seal surface into the container contents and/or from the container contents into the container or seal surface, and to prevent gas and/or water permeability in medical articles, such as syringes, tubes, and medical collection devices.

### SUMMARY OF THE INVENTION

According to some non-limiting embodiments or aspects, a medical device includes a container defined by walls and having a first end having an opening, where the opening is sealed by a rubber component, where the rubber component is formed from a self-lubricating rubber formed from a rubber composition including a halogenated isobutylene-isoprene co-polymer and at least one of (1) clay minerals or silica and/or (2) a liquid polyisoprene and butadiene homopolymer.

The medical device may be selected from the group consisting of a syringe assembly, drug cartridge, needleless injector, liquid dispensing device, liquid metering device, sample collection tube or plate assembly, catheter, and vial. The rubber component may include a pierceable septum. The medical device may include a drug delivery system for injecting a medicament, where the container includes a syringe barrel configured to receive the medicament, where the rubber component includes a stopper configured to slide against the walls within the syringe barrel from a pre-use position to a post-use position. The halogenated isobutylene-isoprene co-polymer may include a chloro-isobutylene-isoprene co-polymer. The rubber composition may further include the clay minerals or silica. The clay minerals may include calcined magnesium silicate clay or aluminum silicate clay. The rubber composition may further include the liquid polyisoprene and butadiene homopolymer. The liquid polyisoprene and butadiene homopolymer may have a weight average molecular weight of from 20,000 to 50,000 g/mol. The rubber composition may include: from 50 to 100 phr of the halogenated isobutylene-isoprene co-polymer; from 30 to 60 phr of the clay minerals or silica; and from 3 to 30 phr of the liquid polyisoprene and butadiene homopolymer.

The walls may include glass, stainless steel, or a polymeric material. The polymeric material may include polypropylene and/or a cyclic polyolefin. The stopper and/or the walls may be substantially free of a lubricant. The lubricant may include a silicone oil. The halogenated isobutylene-isoprene co-polymer may be non-staining. The rubber composition may be cured to form the self-lubricating rubber by sulfur vulcanization. At least a portion of the rubber component may be laminated by a polymer including ethylene tetrafluoroethylene (ETFE) and/or polytetrafluoroethylene (PTFE).

According to some non-limiting embodiments or aspects, a rubber component configured for use in a medical device includes the rubber component including a self-lubricating rubber formed from a rubber composition including a halogenated isobutylene-isoprene co-polymer and at least one of (1) clay minerals or silica and (2) a liquid polyisoprene and butadiene homopolymer.

The medical device may be selected from the group consisting of a syringe assembly, drug cartridge, needleless injector, liquid dispensing device, liquid metering device, sample collection tube or plate assembly, catheter, and vial. The rubber component may include a stopper and/or a pierceable septum. The rubber component may include a stopper positioned within an opening of a first end of a syringe barrel and configured to slide against an interior wall of the syringe barrel from a pre-use position to a post-use position. The rubber component may include a pierceable septum sealing an opening of a first end of a container. The rubber composition may include: from 50 to 100 phr of the halogenated isobutylene-isoprene co-polymer; from 30 to 60 phr of the clay minerals or silica; and from 3 to 30 phr of the liquid polyisoprene and butadiene homopolymer. The halogenated isobutylene-isoprene co-polymer may include a chloro-isobutylene-isoprene co-polymer. At least a portion of the rubber component may be laminated by a polymer including ethylene tetrafluoroethylene (ETFE) and/or polytetrafluoroethylene (PTFE).

According to some non-limiting embodiments or aspects, a rubber composition includes: from 50 to 100 phr of a halogenated isobutylene-isoprene co-polymer; from 30 to 60 phr of clay minerals or silica; and from 3 to 30 phr of a liquid polyisoprene and butadiene homopolymer.

The halogenated isobutylene-isoprene co-polymer may include a chloro-isobutylene-isoprene co-polymer. The clay minerals may include calcined magnesium silicate clay or aluminum silicate clay. The liquid polyisoprene and butadiene homopolymer may have a weight average molecular weight of from 20,000 to 50,000 g/mol.

According to some non-limiting embodiments or aspects, a method for injecting a medicament includes: engaging a drive assembly of a drug delivery system including a container defined by walls and including the medicament, the container including a stopper configured to slide against the walls within the container from a pre-use position to a post-use position, where engaging the drive assembly causes the stopper to slide against the walls from the pre-use position to the post-use position to deliver the medicament from the container to a patient, where the stopper is formed from a self-lubricating rubber formed from a rubber composition including a halogenated isobutylene-isoprene co-polymer and at least one of (1) clay minerals or silica and (2) a liquid polyisoprene and butadiene homopolymer.

The drive assembly may include a plunger member configured to move the stopper within the container, where engaging the drive assembly includes depressing the plunger member.

According to some non-limiting embodiments or aspects, a method of re-sealing a sealed medical container includes: piercing, with a needle, a septum arranged over an opening of a container to form a puncture in the septum; and withdrawing the needle from the puncture of the septum, where the septum is formed from a self-healing rubber formed from a rubber composition including a halogenated isobutylene-isoprene co-polymer and at least one of (1) clay minerals or silica and (2) a liquid polyisoprene and butadiene homopolymer.

Upon withdrawing the needle from the septum, the puncture formed from the needle piercing the septum may be automatically re-sealed by the self-healing rubber.

Other non-limiting embodiments of the disclosure will be set forth in the following numbered clauses:

Clause 1: A medical device comprising a container defined by walls and having a first end having an opening, wherein the opening is sealed by a rubber component, wherein the rubber component is formed from a self-lubricating rubber formed from a rubber composition comprising a halogenated isobutylene-isoprene co-polymer and at least one of (1) clay minerals or silica and (2) a liquid polyisoprene and butadiene homopolymer.

Clause 2: The medical device of clause 1 selected from the group consisting of a syringe assembly, drug cartridge, needleless injector, liquid dispensing device, liquid metering device, sample collection tube or plate assembly, catheter, and vial.

Clause 3: The medical device of clause 1 or clause 2, wherein the rubber component comprises a pierceable septum.

Clause 4: The medical device of any of clauses 1-3, wherein the medical device comprises a drug delivery system for injecting a medicament, wherein the container comprises a syringe barrel configured to receive the medicament, wherein the rubber component comprises a stopper configured to slide against the walls within the syringe barrel from a pre-use position to a post-use position.

Clause 5: The medical device of any of clauses 1-4, wherein the halogenated isobutylene-isoprene co-polymer comprises a chloro-isobutylene-isoprene co-polymer.

Clause 6: The medical device of any of clauses 1-5, wherein the rubber composition further comprises the clay minerals or silica.

Clause 7: The medical device of clause 6, wherein the clay minerals comprise calcined magnesium silicate clay or aluminum silicate clay.

Clause 8: The medical device of any of clauses 1-7, wherein the rubber composition further comprises the liquid polyisoprene and butadiene homopolymer.

Clause 9: The medical device of any of clauses 1-8, wherein the liquid polyisoprene and butadiene homopolymer has a weight average molecular weight of from 20,000 to 50,000 g/mol.

Clause 10: The medical device of any of clauses 1-9, wherein the rubber composition comprises: from 50 to 100 phr of the halogenated isobutylene-isoprene co-polymer; from 30 to 60 phr of the clay minerals or silica; and from 3 to 30 phr of the liquid polyisoprene and butadiene homopolymer.

Clause 11: The medical device of any of clauses 1-10, wherein the walls comprise glass, stainless steel, or a polymeric material.

Clause 12: The medical device of clause 11, wherein the polymeric material comprises polypropylene and/or a cyclic polyolefin.

Clause 13: The medical device of any of clauses 4-12, wherein the stopper and/or the walls are substantially free of a lubricant.

Clause 14: The medical device of clause 13, wherein the lubricant comprises a silicone oil.

Clause 15: The medical device of any of clauses 1-14, wherein the halogenated isobutylene-isoprene co-polymer is non-staining.

Clause 16: The medical device of any of clauses 1-15, wherein the rubber composition is cured to form the self-lubricating rubber by sulfur vulcanization.

Clause 17: The medical device of any of clauses 1-16, wherein at least a portion of the rubber component is laminated by a polymer comprising ethylene tetrafluoroethylene (ETFE) and/or polytetrafluoroethylene (PTFE).

Clause 18: A rubber component configured for use in a medical device, the rubber component comprising a self-lubricating rubber formed from a rubber composition comprising a halogenated isobutylene-isoprene co-polymer and at least one of (1) clay minerals or silica and (2) a liquid polyisoprene and butadiene homopolymer.

Clause 19: The rubber component of clause 18, wherein the medical device is selected from the group consisting of a syringe assembly, drug cartridge, needleless injector, liquid dispensing device, liquid metering device, sample collection tube or plate assembly, catheter, and vial.

Clause 20: The rubber component of clause 18 or clause 19, wherein the rubber component comprises a stopper and/or a pierceable septum.

Clause 21: The rubber component of any of clauses 18-20, wherein the rubber component comprises a stopper positioned within an opening of a first end of a syringe barrel and configured to slide against an interior wall of the syringe barrel from a pre-use position to a post-use position.

Clause 22: The rubber component of any of clauses 18-21, wherein the rubber component comprises a pierceable septum sealing an opening of a first end of a container.

Clause 23: The rubber component of any of clauses 18-22, wherein the rubber composition comprises: from 50 to 100 phr of the halogenated isobutylene-isoprene co-polymer; from 30 to 60 phr of the clay minerals or silica; and from 3 to 30 phr of the liquid polyisoprene and butadiene homopolymer.

Clause 24: The rubber component of any of clauses 18-23, wherein the halogenated isobutylene-isoprene co-polymer comprises a chloro-isobutylene-isoprene co-polymer.

Clause 25: The rubber component of any of clauses 18-24, wherein at least a portion of the rubber component is laminated by a polymer comprising ethylene tetrafluoroethylene (ETFE) and/or polytetrafluoroethylene (PTFE).

Clause 26: A rubber composition comprising: from 50 to 100 phr of a halogenated isobutylene-isoprene co-polymer; from 30 to 60 phr of clay minerals or silica; and from 3 to 30 phr of a liquid polyisoprene and butadiene homopolymer.

Clause 27: The rubber composition of clause 26, wherein the halogenated isobutylene-isoprene co-polymer comprises a chloro-isobutylene-isoprene co-polymer.

Clause 28: The rubber composition of clause 26 or clause 27, wherein the clay minerals comprise calcined magnesium silicate clay or aluminum silicate clay.

Clause 29: The rubber composition of any of clauses 26-28, wherein the liquid polyisoprene and butadiene homopolymer has a weight average molecular weight of from 20,000 to 50,000 g/mol.

Clause 30: A method for injecting a medicament, comprising: engaging a drive assembly of a drug delivery system comprising a container defined by walls and comprising the medicament, the container comprising a stopper configured to slide against the walls within the container from a pre-use position to a post-use position, wherein engaging the drive assembly causes the stopper to slide against the walls from the pre-use position to the post-use position to deliver the medicament from the container to a patient, wherein the stopper is formed from a self-lubricating rubber formed from a rubber composition comprising a halogenated isobutylene-isoprene co-polymer and at least one of (1) clay minerals or silica and (2) a liquid polyisoprene and butadiene homopolymer.

Clause 31: The method of clause 30, wherein the drive assembly comprises a plunger member configured to move the stopper within the container, wherein engaging the drive assembly comprises depressing the plunger member.

Clause 32: A method of re-sealing a sealed medical container comprising: piercing, with a needle, a septum arranged over an opening of a container to form a puncture in the septum; and withdrawing the needle from the puncture of the septum, wherein the septum is formed from a self-healing rubber formed from a rubber composition comprising a halogenated isobutylene-isoprene co-polymer and at least one of (1) clay minerals or silica and (2) a liquid polyisoprene and butadiene homopolymer.

Clause 33: The method of clause 32, wherein upon withdrawing the needle from the septum, the puncture formed from the needle piercing the septum is automatically re-sealed by the self-healing rubber.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a medical device according to some non-limiting embodiments or aspects.

FIG. 2 is a perspective, cross-sectional view of the medical device of FIG. 1 according to some non-limiting embodiments or aspects.

FIG. 3 is a front, cross-sectional view of the medical device of FIG. 1 according to some non-limiting embodiments or aspects.

FIG. 4 is a top view of the medical device of FIG. 1 showing a top portion of the housing removed and the medical device (in the form of a drug delivery system) in a pre-use position according to some non-limiting embodiments or aspects.

FIG. 5 is a top, cross-sectional view of the medical device of FIG. 1 showing the drug delivery system in a pre-use position according to some non-limiting embodiments or aspects.

FIG. 6 is a front, cross-sectional view of the medical device of FIG. 1 showing the drug delivery system in a pre-use position according to some non-limiting embodiments or aspects.

FIG. 7 is a top view of the medical device of FIG. 1 showing a top portion of the housing removed and the drug delivery system in an initial actuation position according to some non-limiting embodiments or aspects.

FIG. 8 is a top, cross-sectional view of the medical device of FIG. 1 showing the drug delivery system in an initial actuation position according to some non-limiting embodiments or aspects.

FIG. 9 is a front, cross-sectional view of the medical device of FIG. 1 showing the drug delivery system in an initial actuation position according to some non-limiting embodiments or aspects.

FIG. 10 is a top view of the medical device of FIG. 1 showing a top portion of the housing removed and the drug delivery system in a use position according to some non-limiting embodiments or aspects.

FIG. 11 is a top, cross-sectional view of the medical device of FIG. 1 showing the drug delivery system in a use position according to some non-limiting embodiments or aspects.

FIG. 12 is a front, cross-sectional view of the medical device of FIG. 1 showing the drug delivery system in a use position according to some non-limiting embodiments or aspects.

FIG. 13 is a top view of the medical device of FIG. 1 showing a top portion of the housing removed and the drug delivery system in a post-use position according to some non-limiting embodiments or aspects.

FIG. 14 is a top, cross-sectional view of the medical device of FIG. 1 according to some non-limiting embodiments or aspects showing the drug delivery system in a post-use position according to some non-limiting embodiments or aspects.

FIG. 15 is a front, cross-sectional view of the medical device of FIG. 1 showing the drug delivery system in a post-use position according to some non-limiting embodiments or aspects.

FIG. 15A is a front, cross-sectional view of the medical device of FIG. 1 showing a pad with the drug delivery system in a pre-use position according to some non-limiting embodiments or aspects.

FIG. 15B is a perspective, cross-sectional view of the medical device of FIG. 1 showing a pad with the drug delivery system in a pre-use position according to some non-limiting embodiments or aspects.

FIG. 15C is a perspective, cross-sectional view of the medical device of FIG. 1 showing a pad with the drug delivery system in a pre-use position according to some non-limiting embodiments or aspects.

FIG. 16 is a partial cross-sectional view of the medical device of FIG. 1 showing a valve assembly according to some non-limiting embodiments or aspects.

FIG. 17 is perspective view of a medical device according to some non-limiting embodiments or aspects.

FIG. 18 is a perspective view of the medical device of FIG. 17 showing a top portion of a housing removed according to some non-limiting embodiments or aspects.

FIG. 19 is a top view of the medical device of FIG. 17 showing the medical device in a pre-use position according to some non-limiting embodiments or aspects.

FIG. 20 is a top view of the medical device of FIG. 17 showing the medical device in a use position according to some non-limiting embodiments or aspects.

FIG. 21 shows activation and gliding forces (AGF) test results for Examples 6-8.

FIG. 22 shows peak and slip coefficients test results for Examples 6 and 8.

FIG. 23 shows DMA-TTS test results for Examples 6 and 8.

FIG. 24 shows peel-off test results for Examples 6 and 8.

Corresponding reference characters indicate corresponding parts throughout the several views. The exemplifications set out herein illustrate exemplary aspects of the disclosure, and such exemplifications are not to be construed as limiting the scope of the disclosure in any manner.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

Other than in the operating examples, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements. Furthermore, when numerical ranges of varying scope are set forth herein, it is contemplated that any combination of these values inclusive of the recited values may be used.

Also, it should be understood that any numerical range recited herein is intended to include all sub-ranges subsumed therein. For example, a range of “1 to 10” is intended to include all sub-ranges between and including the recited minimum value of 1 and the recited maximum value of 10, that is, having a minimum value equal to or greater than 1 and a maximum value of equal to or less than 10. A range of “less than 5” includes all subranges below 5.

The present disclosure is directed to a medical device comprising a container defined by walls and having a first end having an opening, wherein the opening is sealed by a rubber component, wherein the rubber component is formed from a self-lubricating rubber formed from a rubber composition comprising a halogenated isobutylene-isoprene co-polymer and at least one of (1) clay minerals or silica and (2) a liquid polyisoprene and butadiene homopolymer.

The medical device may be selected from the group consisting of a syringe assembly, drug cartridge, needleless injector, liquid dispensing device, liquid metering device, sample collection tube or plate assembly, catheter, and vial, although it may be appreciated that other medical devices may be used. As used herein, “medical device” means any article of manufacture that can be useful for medical treatment.

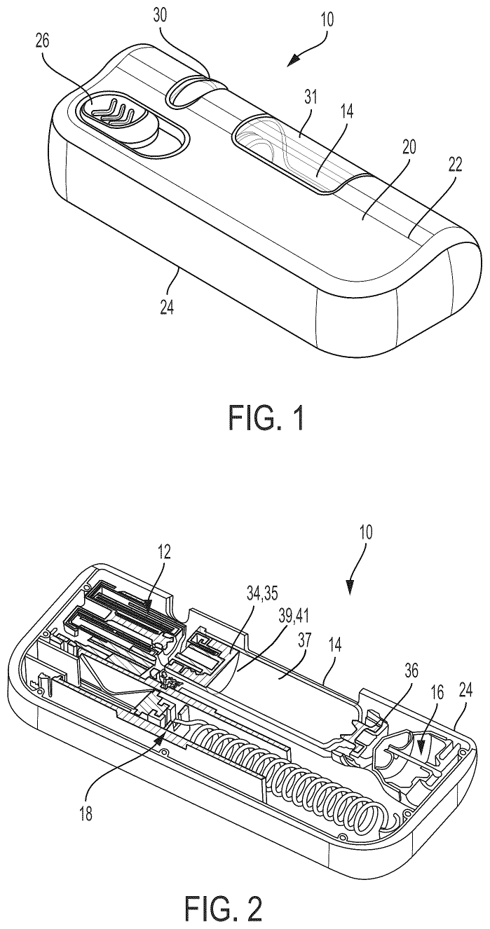

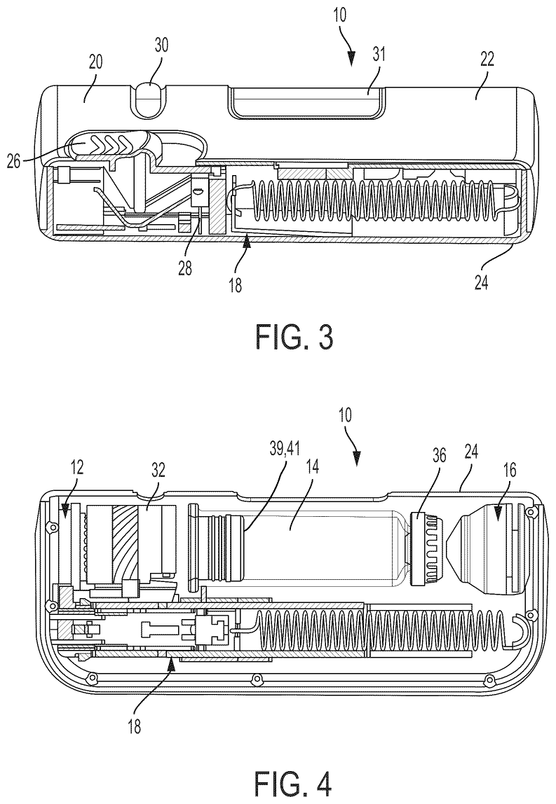

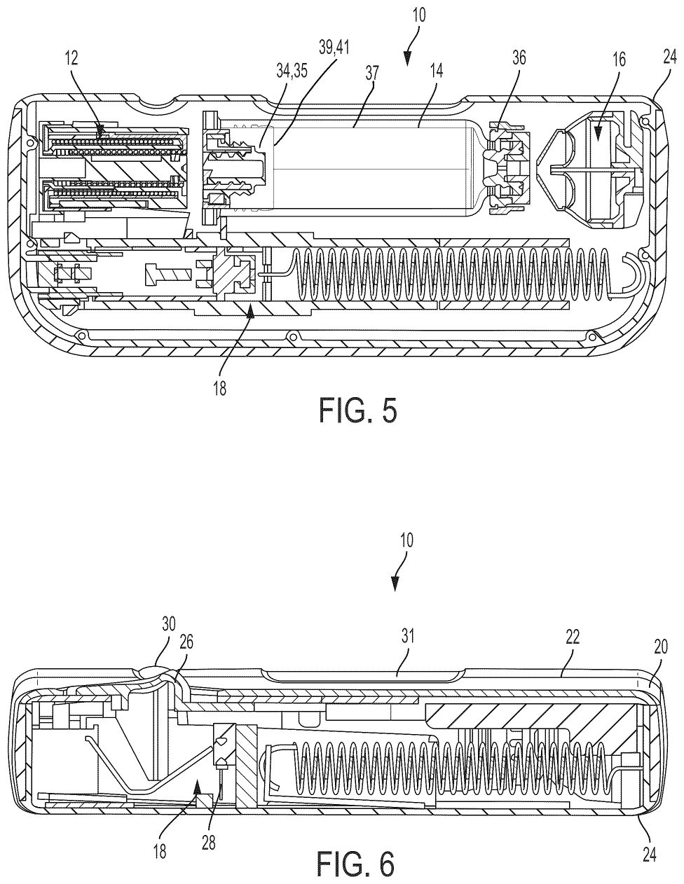

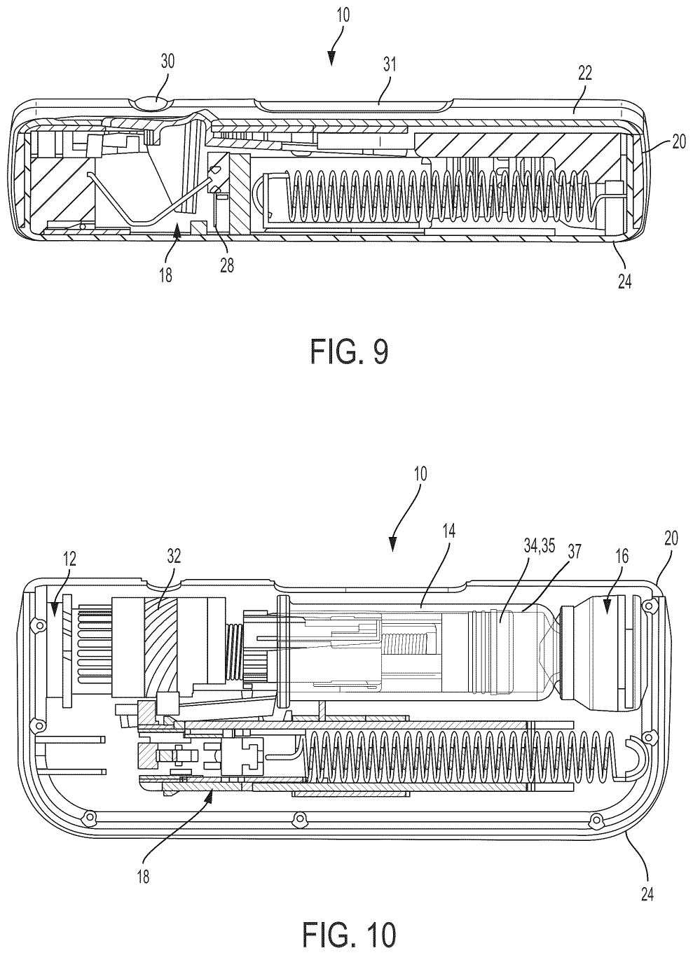

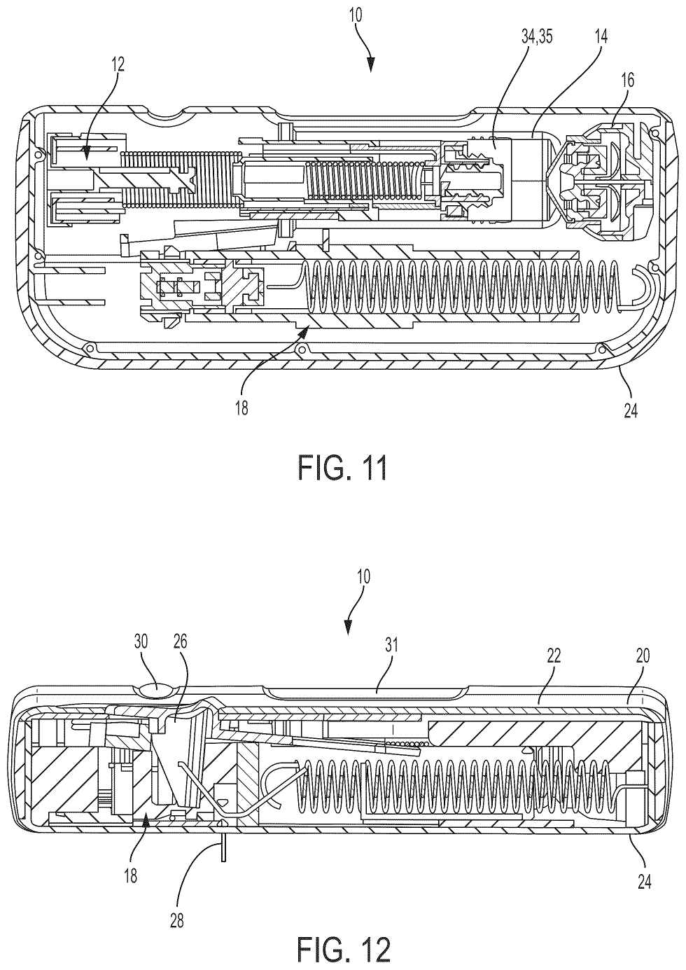

Non-limiting examples of medical devices that may be used according to the present disclosure include the drug delivery systems (medical devices) 10, 200 shown and described in connection with FIGS. 1-20.

Referring to FIGS. 1-16, a drug delivery system 10 according to some non-limiting embodiments or aspects may include a drive assembly 12, a container 14, a valve assembly 16, and a needle actuator assembly 18. The drive assembly 12, the container 14, the valve assembly 16, and the needle actuator assembly 18 may be at least partially positioned within a housing 20. The housing 20 may include a top portion 22 and a bottom portion 24, although other suitable arrangements for the housing 20 may be utilized. In one aspect, the drug delivery system 10 may be an injector device configured to be worn or secured to a user and to deliver a predetermined dose of a medicament provided within the container 14 via injection into the user. The system 10 may be utilized to deliver a “bolus injection” where a medicament is delivered within a set time period. The medicament may be delivered over a time period of up to 45 minutes, although other suitable injection amounts and durations may be utilized. A bolus administration or delivery can be carried out with rate controlling or have no specific rate controlling. The system 10 may deliver the medicament at a fixed pressure to the user with the rate being variable. The general operation of the system 10 is described below in reference to FIGS. 1-16 with the specifics of the drive assembly 12 and needle actuator assembly 18.

Referring again to FIGS. 1-16, the system 10 may be configured to operate through the engagement of an actuation button 26 by a user, which results in a needle 28 of the needle actuator assembly 18 piercing the skin of a user, the actuation of the drive assembly 12 to place the needle 28 in fluid communication with the container 14 and to expel fluid or medicament from the container 14, and the withdrawal of the needle 28 after injection of the medicament is complete. The general operation of a drug delivery system is shown and described in International Publication Nos. 2013/155153 and 2014/179774, which are hereby incorporated by reference in their entirety. The housing 20 of the system 10 may include an indicator window 30 for viewing an indicator arrangement 32 configured to provide an indication to a user on the status of the system 10 and a container window 31 for viewing the container 14. The indicator window 30 may be a magnifying lens for providing a clear view of the indicator arrangement 32. The indicator arrangement 32 may move along with the needle actuator assembly 18 during use of the system 10 to indicate pre-use status, use status, and post-use status of the system 10. The indicator arrangement 32 may provide visual indicia regarding the status, although other suitable indicia, such an auditory or tactile, may be provided as an alternative or additional indicia.

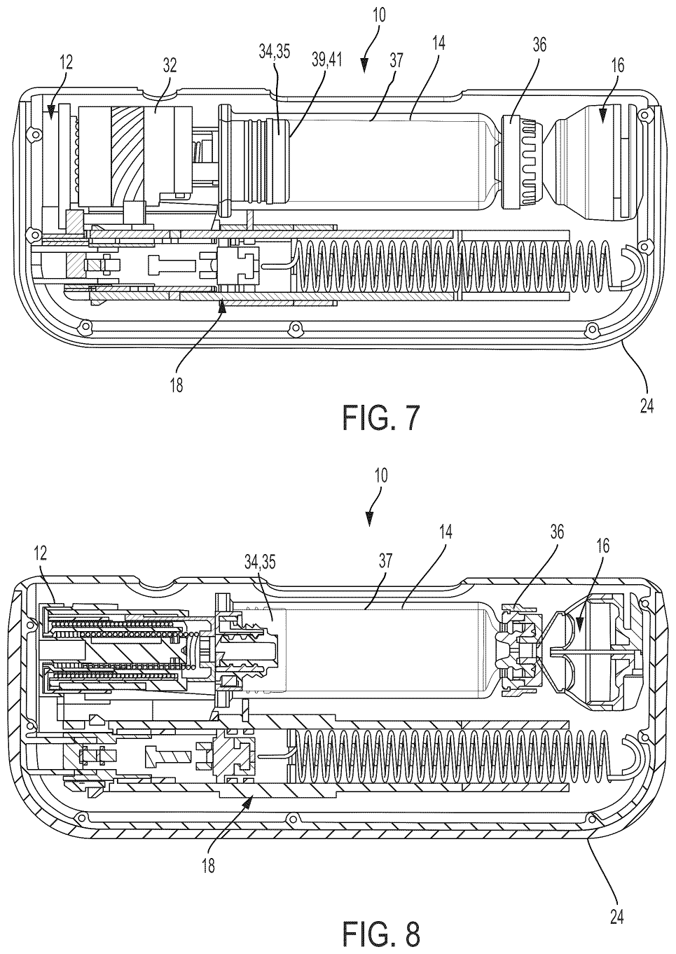

Referring to FIGS. 4-6, during a pre-use position of the system 10, the container 14 may be spaced from the drive assembly 12 and the valve assembly 16 and the needle 28 may be in a retracted position. During the initial actuation of the system 10, as shown in FIGS. 7-9, the drive assembly 12 may engage the container 14 to move the container 14 toward the valve assembly 16, which is configured to pierce a closure 36 of the container 14 and place the medicament within the container 14 in fluid communication with the needle 28 via a tube (not shown) or other suitable arrangement. The drive assembly 12 may be configured to engage a stopper 34 of the container 14, which will initially move the entire container 14 into engagement with the valve assembly 16 due to the incompressibility of the fluid or medicament within the container 14. The initial actuation of the system 10 may be caused by engagement of the actuation button 26 by a user, which releases the needle actuator assembly 18 and the drive assembly 12 as discussed below in more detail. During the initial actuation, the needle 28 may still be in the retracted position and about to move to the extended position to inject the user of the system 10.

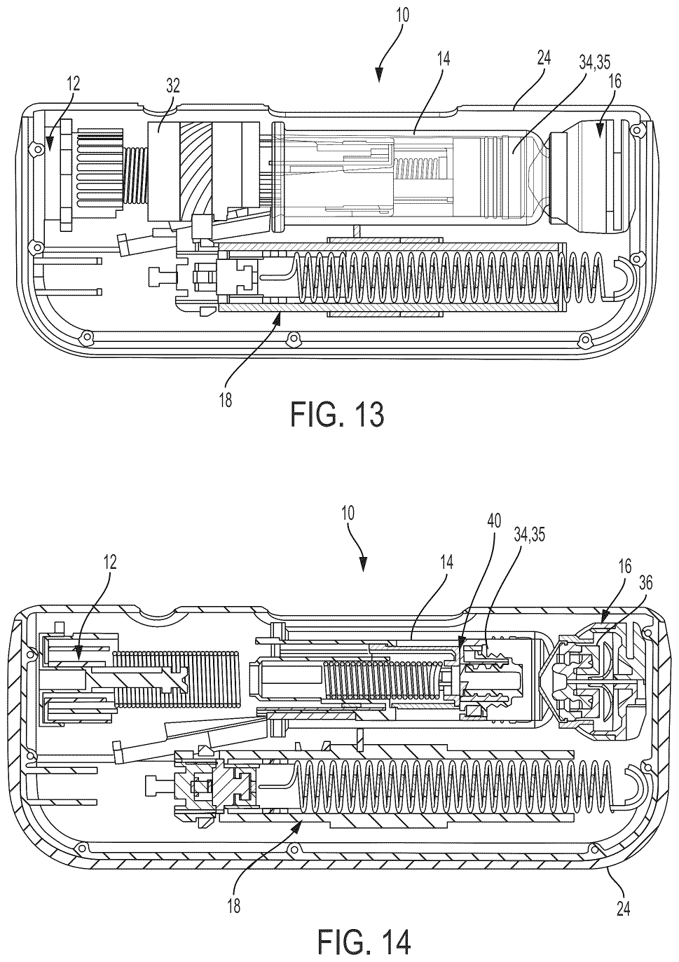

During the use position of the system 10, as shown in FIGS. 10-12, the needle 28 may be in the extended position at least partially outside of the housing 20 with the drive assembly 12 moving the stopper 34 within the container 14 to deliver the medicament from the container 14, through the needle 28, and to the user. In the use position, the valve assembly 16 may have already pierced a closure 36 of the container 14 to place the container 14 in fluid communication with the needle 28, which also allows the drive assembly 12 to move the stopper 34 relative to the container 14 since fluid is able to be dispensed from the container 14. At the post-use position of the system 10, shown in FIGS. 13-15, the needle 28 may be in the retracted position and engaged with a pad 38 to seal the needle 28 and prevent any residual flow of fluid or medicament from the container 14. The container 14 and valve assembly 16 may be the container 14 and valve assembly 16 shown and described in International Publication No. WO 2015/081337, which is hereby incorporated by reference in its entirety.

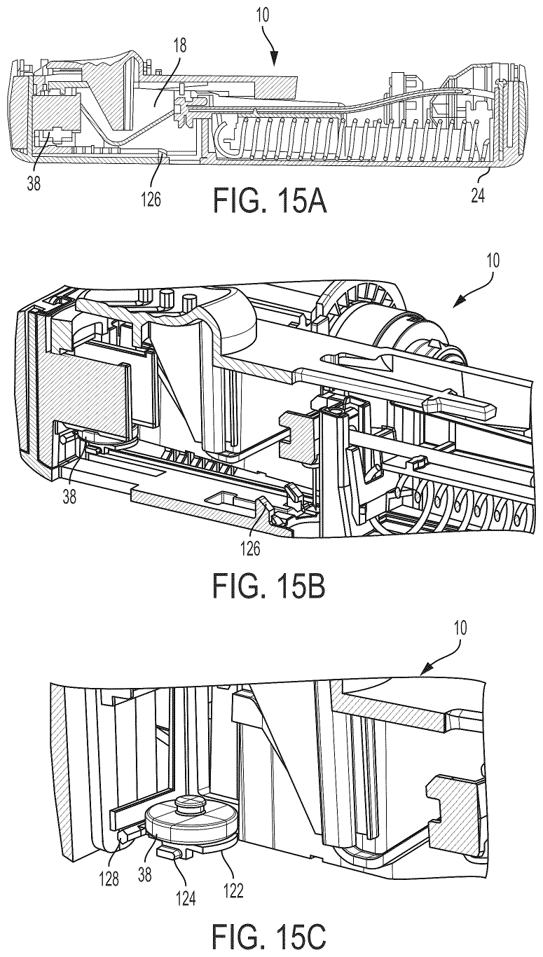

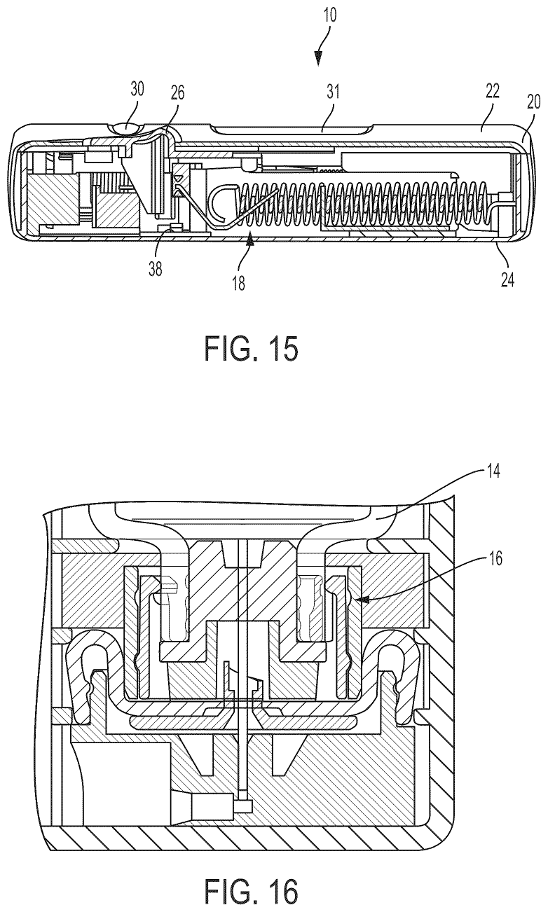

Referring to FIGS. 15A-15C, the pad 38 may be biased into the pad as the system moves from the use position to the post-use position. In particular, the pad 38 may be received by a pad arm 122 having a cam surface 124 that cooperates with a cam track 126 on the bottom portion 24 of the housing 20. The pad arm 122 may be connected to a needle actuator body via a torsion bar 128. The cam surface 124 may be configured to engage the cam track 126 to deflect the pad arm 122 downwards thereby allowing the pad 38 to pass beneath the needle 28 before being biased upwards into the needle 28. The torsion bar 128 allows the pad arm 122 to twist about a pivot of the needle actuator body. The pad 38 may be press-fit into an opening of the pad arm 122, although other suitable arrangements for securing the pad 38 may be utilized.

With continued reference to FIGS. 1-16, the drug delivery system 10 may include a rubber component 35, in the form of a stopper 34 as shown in this non-limiting example. The drug delivery system 10 may comprise the container 14 defined by walls 37. The container 14 may have a first end 39 having an opening 41 sealed by the rubber component 35. In the non-limiting example shown in FIGS. 1-16, the container 14 comprises a syringe barrel configured to receive a medicament. The rubber component 35 (stopper 34) is configured to slide against the walls 37 within the syringe barrel from a pre-use position to post-use position to force the medicament from the container 14, such as to a patient.

The walls 37 of the container 14 may be made of any suitable materials. For example, the walls 37 may be formed from glass, metal, ceramic, plastic, rubber or combinations thereof.

In some non-limiting embodiments, the walls 37 may be prepared from Type I borosilicate glass.

In some non-limiting embodiments, the walls 37 may be prepared from one or more olefinic polymers, such as polyethylene, polypropylene, poly(1-butene), poly(2-methyl-1-pentene), and/or cyclic polyolefins. For example, the polyolefin can be a homopolymer or a copolymer of an aliphatic monoolefin, the aliphatic monoolefin preferably having from 2 to 6 carbon atoms, such as polypropylene. In some non-limiting embodiments, the polyolefin can be basically linear, but optionally may contain side chains such as are found, for instance, in conventional, low density polyethylene. In some non-limiting embodiments, the polyolefin is at least 50% isotactic. In other non-limiting embodiments, the polyolefin is at least about 90% isotactic in structure. In some non-limiting embodiments, syndiotactic polymers can be used.

In some embodiments, cyclic polyolefins can be used. Non-limiting examples of suitable cyclic polyolefins include dicyclopentadiene (DCP), norbornene, tetracyclododecene (TCD), alternating, random or block ethylene/norbonanediyl units, or other polymeric type units such as are disclosed in U.S. Pat. Nos. 6,525,144, 6,511,756, 5,599,882, and 5,034,482 (each of Nippon Zeon), U.S. Pat. Nos. 7,037,993, 6,995,226, 6,908,970, 6,653,424, and 6,486,264 (each of Zeon Corp.), U.S. Pat. Nos. 7,026,401 and 6,951,898 (Ticona), U.S. Pat. No. 6,063,886 (Mitsui Chemicals), U.S. Pat. Nos. 5,866,662, 5,856,414, 5,623,039, and 5,610,253 (Hoechst), U.S. Pat. Nos. 5,854,349 and 5,650,471 (Mitsui Petrochemical and Hoechst) and as described in “Polycyclic olefins”, Process Economics Program (July 1998) SRI Consulting, each of the foregoing references being incorporated by reference herein. Non-limiting examples of suitable cyclic polyolefins include APEL cyclic polyolefins available from Mitsui Petrochemical, TOPAS cyclic polyolefins available from Ticona Engineering Polymers, ZEONOR or ZEONEX cyclic polyolefins available from Zeon Corporation, and cyclic polyolefins available from Promerus LLC.

The polyolefin can contain a small amount, generally from 0.1 to 10 weight percent, of an additional polymer incorporated into the composition by copolymerization with the appropriate monomer. Such copolymers may be added to the composition to enhance other characteristics of the final composition, and may be, for example, polyacrylate, polystyrene, and the like.

In some non-limiting embodiments, the walls 37 may be constructed of a polyolefin composition which includes a radiation stabilizing additive to impart radiation stability to the container, such as a mobilizing additive which contributes to the radiation stability of the container, such as for example those disclosed in U.S. Pat. Nos. 4,959,402 and 4,994,552, assigned to Becton, Dickinson and Company and both of which are incorporated herein by reference.

In some non-limiting embodiments, the walls may be of a blood collection medical device. The blood collection device can be either an evacuated blood collection tube or a non-evacuated blood collection tube. The blood collection tube can be made of polyethylene terephthalate, polypropylene, polycarbonate, polycycloolefin, polyethylene naphthalate or copolymers thereof.

In some non-limiting embodiments, the walls 37 may be prepared from stainless steel.

The dimensions, e.g., inner and outer diameter, length, wall thickness, etc. of the container 14 can be of any size desired. For example, for a one ml volume syringe barrel, the inner diameter of the barrel may be about 0.25 inches (6.35 mm) and the length may be about 2.0 inches (50.8 mm). For a plastic Sterifill 20 ml volume syringe barrel, the inner diameter of the barrel may be about 0.75 inches (19.05 mm) and the length may be about 3.75 inches (95.3 mm). Generally, the inner diameter can range from 0.25 inches (6.35 mm) to 10 inches (254 mm), or from 0.25 inches (6.35 mm) to 5 inches (127 mm), or any value therebetween.

With continued reference to FIGS. 1-16, the rubber component 35 and the walls 37 may be substantially free of a lubricant, due to the rubber component 35 comprising the self-lubricating rubber as described herein. As used herein “substantially free of a lubricant” indicates that a lubricant is not intentionally added to the rubber component 35 and the walls 37, such that any trace amounts of lubricant present are incidental. The rubber component 35 and the walls 37 may be completely free of a lubricant. Non-limiting examples of lubricants include silicone or hydrocarbon oils, such as mineral oils, peanut oil, vegetable oils, and the like.

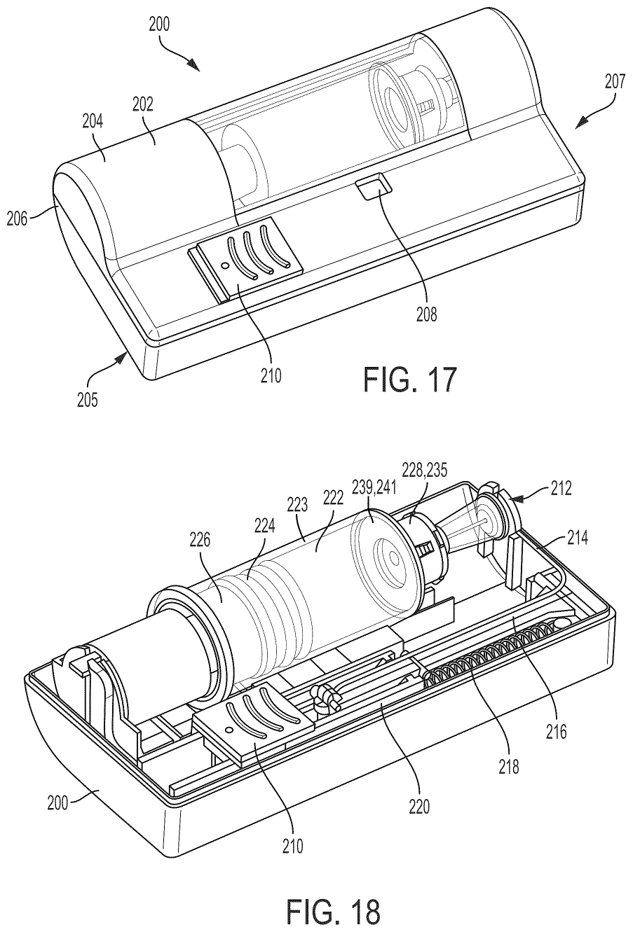

Referring to FIGS. 17-20, a drug delivery system 200 according to a further non-limiting embodiment is shown. The system 200 may include a housing 202 having an upper housing 204 and a lower housing 206. The housing may have a proximal end 205 and a distal end 207. The upper housing 204 may have a status view port 208 so that a user can view the operating status of the system 200. The system 200 may also include a valve assembly 212 and a tube 214 fluidly connecting the valve assembly 212 with a patient needle that is disposed in a proximal end of a needle arm 216. A user may slide the actuation button 210 proximally and then displaces the button 210 vertically into the housing 202, thereby freeing the needle actuator 220 to displace distally under the influence of the spring 218.

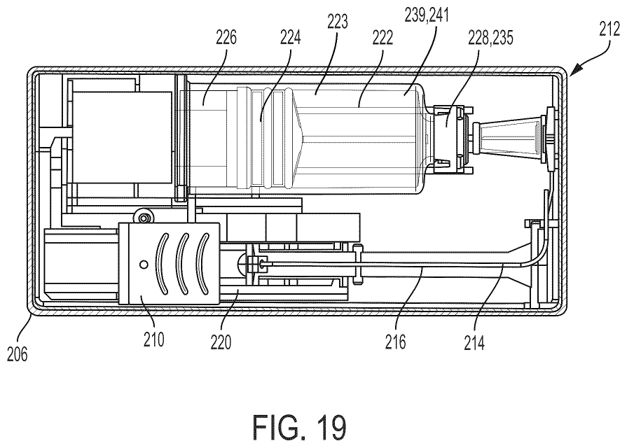

As shown in FIG. 18, the system 200 may additionally include a container or medicament container 222 with a stopper 224 movably disposed therein. Preferably, the distal end of the medicament container 222 has a septum 228 that is spaced apart from the valve assembly 212 prior to actuation of the device, as best shown in FIG. 19.

For manufacturing purposes, using one size for a medicament container is often desirable, even if multiple fill volumes or dosages are contemplated for use with the container. In such cases, when medicament containers are filled, the differing fill volumes result in different positions of the stopper. To accommodate such different stopper positions, as well as accommodate manufacturing differences of the stoppers, aspects of the present disclosure may include a bespoke or custom spacer 226 disposed in a proximal end of the container 222, proximal to the stopper 224. In other words, the bespoke spacer 226 provides an option that allows dispensing of a range of manufacturer-set pre-defined fill volumes by selection of different spacers 226, and reduces or eliminates the need for assembly configuration operations. The size of the spacer 226 can be employed to account for under-filled volumes of the container 222, and provide a consistent bearing surface at the proximal end of the container.

The spacer 226 may be selected from a plurality of different size spacers 226 to occupy space from a proximal end of the stopper 224 to a proximal end of the container 222. According to one embodiment, as shown in FIG. 19, the spacer 226 is selected to be substantially flush with the proximal end of the container 222.

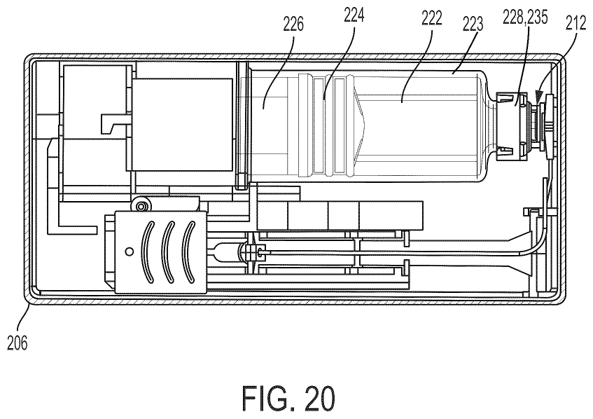

Referring to FIG. 20 a plunger (not shown) may be distally driven to contact the spacer 226. Because the medicament container 222 is filled with a substantially incompressible fluid, the continued distal displacement of the plunger distally displaces the spacer 226, the stopper 224, and the container 222 relative to the housing 202. This distal displacement may cause the septum 228 to be pierced by the valve assembly 212, establishing fluid communication between the container 222 and the patient needle.

With continued reference to FIGS. 17-20, the drug delivery system 200 may include a rubber component 235, in the form of a septum 228 as shown in this non-limiting example. The drug delivery system 200 may comprise the container 222 defined by walls 223. The container 222 may have a first end 239 having an opening 241 sealed by the rubber component 235. In the non-limiting example shown in FIGS. 17-20, the container 222 may comprise a syringe barrel configured to receive a medicament. The rubber component 235 (septum 228) may seal the first end 239 of the container 222.

The rubber component 235 may comprise a pierceable septum 228 pierceable by a needle, such as a needle of the valve assembly 212. The stopper 224 may also be a rubber component.

Although the medical device of the present disclosure has been described in detail in connection with the medical devices shown in FIGS. 1-20, it will be appreciated that other medical devices may be used that contain the rubber component as described herein. The rubber component as described herein may be a stopper, a septum, or other rubber component (e.g., O-ring, V-ring, plunger tip, piston, etc.) included in the medical device. The rubber component may comprise an intermediate stopper in a multiple chambered syringe or drug container, such as a sequential stopper or a bypass stopper.

The rubber component configured for use in a medical device may comprise a self-lubricating rubber. The self-lubricating rubber may be formed from a rubber composition comprising a halogenated isobutylene-isoprene co-polymer. In some non-limiting embodiments or aspects, the halogenated isobutylene-isoprene co-polymer may comprise a chloro-isobutylene-isoprene co-polymer and/or a bromo-isobutylene-isoprene co-polymer (BIIR), such as and brominated isobutylene paramethyl-styrene terpolymers. The rubber composition may comprise from 50 to 100 phr, such as 70 to 100 phr, of the halogenated isobutylene-isoprene co-polymer. The term “phr” as used herein, and according to conventional practice, refers to parts by weight of a respective material per 100 parts by weight of rubber.

The halogenated isobutylene-isoprene co-polymer may be non-staining such that its physical contact with other components (e.g., the walls of the medical device) does not stain the other components.

The rubber composition may further comprise clay minerals or silica. For example, the clay minerals may comprise of treated and/or untreated calcined magnesium silicate clay or aluminum silicate clay. The rubber composition may comprise from 30 to 60 phr of clay minerals or silica.

The rubber composition may further comprise a liquid polyisoprene and butadiene homopolymer, non-limiting examples include LIR 30, LIR 290, LIR 390 (polyisoprene liquid rubber) and LBR 305 (butadiene liquid rubber), available from Kuraray America, Inc. (Houston, TX). The liquid polyisoprene and butadiene homopolymer may have a weight average molecular weight of from 20,000 to 50,000 g/mol, as determined using gel permeation chromatography (GPC). The rubber composition may comprise from 3 to 30 phr of the liquid polyisoprene and butadiene homopolymer. The liquid polyisoprene and butadiene homopolymer may enhance the viscoelastic properties of the rubber component and the self-lubricating mechanism thereof.

The rubber composition may comprise the halogenated isobutylene-isoprene co-polymer, the clay minerals or silica, and the liquid polyisoprene and butadiene homopolymer, such as in the amount of from 50 to 100 phr of the halogenated isobutylene-isoprene co-polymer, from 30 to 60 phr of the clay minerals or silica, and from 3 to 30 phr of the liquid polyisoprene and butadiene homopolymer.

The rubber composition may further comprise other pertinent additives for curing and processing of the rubber composition. For example, the pertinent additives may comprise the agents used to vulcanize the rubber composition as described herein.

The rubber composition may be cured to form the self-lubricating rubber. The rubber composition may be cured to form the self-lubricating rubber using a sulfur vulcanization process. The sulfur vulcanization process may comprise using primary sulfur donor accelerators, secondary sulfur donor accelerators, or a blend thereof activated by a metallic oxide. The vulcanization process may form crosslinks between the polymer and sulfur to enhance the strength of the rubber. The rubber may be subjected to suitable heat and/or pressure conditions for a suitable time to form the crosslinks. The rubber composition may be a thermoset rubber composition.

The rubber component may be laminated or unlaminated. The laminated rubber component may be fully laminated (covering the entire rubber component) or partially laminated (having laminated and unlaminated regions).

The rubber component may be laminated by applying a coating composition thereto. The coating composition may comprise ethylene tetrafluoroethylene (ETFE) and/or polytetrafluoroethylene (PTFE). The coating composition may consist essentially of ETFE and/or PTFE. The coating composition may comprise a polydimethylsiloxane. The coating composition may comprise one or more polymers selected from the group of ultra-high molecular weight poly(ethylene) (“UHMWPE”), poly(vinylidene fluoride) (“PVF”), poly(amide), poly(propylene), poly(p-phenylene vinylene) (“PPV”), poly(p-phenylene sulfide) (“PPS”) and combinations thereof. The coating composition may comprise an organosilicon.

The coating composition applied to the rubber component may be dried and/or cured to form a lamination layer thereon. The thickness of the lamination layer formed by applying the coating composition may range from 10 nm to 20 μm, or from 500 nm to 1000 nm, or from 1000 nm to 20 μm.

Alternatively, the rubber component may be unlaminated so as to be free of any lamination layer. Unlaminated rubber components allow for the reduction in the use of extractable or leachable components used in the preparation of the medical device.

The rubber component may exhibit a peak coefficient between glass and the rubber of less than 0.7 lb/lb (measured using a methodology based on ASTM D1894: an Ares G2 rotational shear rheometer was used using a modified Tribo-Rheometry accessory where the rubber, based on rubber sheets molded IAW ASTM D3182 was tested against float glass manufactured as per U.S. Standard MIL-PRF-13830B), a Shore A hardness of from 45-55 units (measured according to IAW ASTM D2240), a compression set after conditioning at room temperature for 24 to 72 hours of up to 45% (measured according to IAW ASTM D395), and a creep strain relaxation Arrhenius's factor of approximately 15,000 to 25,000 under a 0.35 MPa stress at temperatures from 0° C. to 70° C.

When in use in a stopper application, the rubber component may exhibit a breakloose force of less than 50 N, such as less than 40 N, less than 30 N, less than 20 N, or less than 15 N, and a gliding force of less than 25 N, such as less than 20 N, less than 15 N, less than 10 N, or less than 5 N, as determined when tested in a 1 ml long glass syringe with dimensions and test methodology as per ISO 11040.

The rubber component formed from the rubber composition may comprise a stopper and/or a pierceable septum, or other rubber component of a medical device. For example, the rubber component may comprise a stopper positioned within an opening of a first end of a syringe barrel and configured to slide against an interior wall of the syringe barrel from a pre-use position to a post-use position, as shown and described in FIGS. 1-16. For example, the rubber component may comprise a pierceable septum sealing an opening of a first end of a container, as shown and described in FIGS. 17-20.

The present disclosure is also directed to a method for injecting a medicament using the medical device described herein. The medical device may comprise a drug delivery system, such as the syringe shown in FIGS. 1-20. The method may include engaging a drive assembly of a drug delivery system comprising a container defined by walls and comprising the medicament. The container may comprise a stopper configured to slide against the walls within the container from a pre-use position to a post-use position. Engaging the drive assembly causes the stopper to slide against the walls from the pre-use position to the post-use position to deliver the medicament from the container to a patient. The stopper may be formed from the self-lubricating rubber described herein, which is formed from the rubber composition comprising the halogenated isobutylene-isoprene co-polymer and at least one of (1) the clay minerals or silica and (2) the liquid polyisoprene and butadiene homopolymer. For example, engaging the drive assembly may comprise depressing a plunger member configured to move the stopper within the container.

The present disclosure is also directed to a method for re-sealing a sealed medical container. The method may include piercing, with a needle, a septum (the rubber component) arranged over an opening of a container to form a puncture in the septum. The method may include withdrawing the needle from the puncture of the septum. The septum may be formed from the self-lubricating (and/or self-healing) rubber described herein, which is formed from the rubber composition comprising the halogenated isobutylene-isoprene co-polymer and at least one of (1) the clay minerals or silica and (2) the liquid polyisoprene and butadiene homopolymer. Upon withdrawing the needle from the septum, the puncture formed from the needle piercing the septum may be automatically resealed by the rubber component being self-healing. The resealed rubber component may be resealed to the extent so as to act as a barrier component to reduce permeability to oxygen and other gases.

The inclusion for the rubber component from the self-lubricating rubber in the medical device may reduce the friction coefficient between the rubber component and a surface of the medical device against which the rubber component is configured to slide. Quantifiably, the rubber component described herein may have at least an order of magnitude lower coefficient of friction and a lower break free (or breakloose) force than other available medical stopper products. The enhanced slideability of the rubber component, such as in stopper applications, can be achieved without external lubricants (e.g., silicone oil), thus avoiding the inclusion of materials that can have deleterious effects over the active pharmaceutical ingredient (API) or trigger immunogenetic reactions.

The rubber component formed as disclosed herein was surprisingly found to have a viscoelastic response that does not show a glassy state plateau consistent with many elastomeric materials at low temperatures (e.g., −90° C.) but still shows a rubber behavior consistent with elastomer systems at higher temperatures and strains, which shows suitability of the rubber at high frequencies during friction (such as those encountered in septum applications) as well as in ultra-cold storage conditions having temperatures below the thermal and viscoelastic Tg. These unexpected properties also have implications in providing good seals, as the glassy plateau at ultra-low temperatures from other elastomeric materials results in poor sealing between the rubber component and the rigid substrate, resulting in loss of internal components in a pre-filled syringe system.

The rubber component used in medical devices described herein also has a low gas permeability, such as a lower permeability to oxygen and moisture, such that it is suitable for medical device applications in which very low weight losses and maintaining vacuum levels are desired, such as in pre-filled drug products and blood draw tubes.

When used for septum and sealing applications, the rubber component described herein allows for re-sealing upon penetration of a needle without coring or fragmentation.

The rubber component described herein also exhibits good adhesion to fluorinated films, such that the rubber component is suitable for application in contact with biotechnology drug products requiring very low leachables, chemotherapy aggressive medical treatments and drugs, and other systems exposed to harsh conditions.

The present disclosure is more particularly described in the following examples, which are intended to be illustrative only, as numerous modifications and variations therein will be apparent to those skilled in the art.

### EXAMPLES

### Examples 1-5

### Testing of Rubber Stoppers

10 mL size rubber stoppers were prepared according to the present disclosure. The rubber stoppers were prepared by mixing the components from Table 1 below. The lamination for Examples 2, 4, and 5 was performed simultaneously with molding of the rubber.

TABLE 1

| Component | PHR |
|---|---|
| Chloro-isobutylene- | 95 |
| isoprene co-polymer | |
| Semi Crystalline Polyolefin | 5 |
| LIR 30² | 5 |
| LIR 290³ | 5 |
| LBR 305⁴ | 5 |
| PEG 8000⁵ | 10 |
| Mistron Vapor | 40 |
| POLYFIL 70⁶ | 55 |
| Mineral Oil | 7 |
| Paraffin Wax | 2 |
| PE Wax | 7 |
| Stearic Acid | 1.5 |
| ProAid Plus⁷ | 2 |
| Zinc Oxide | 7 |
| Antioxidant DQ⁸ | 2 |
| VULTAC 710⁹ | 3 |
| VULCOFAC CLD 80¹⁰ | 0.5 |
| TBzTD¹¹ | 0.75 |
| Colorants | 1.2 |

²liquid isoprene rubber homopolymer available from Kuraray America, Inc.
³liquid hydrogenated isoprene rubber available from Kuraray America, Inc.
⁴butadiene homopolymer rubber available from Kuraray America, Inc.
⁵poly(ethylene glycol) having an average molecular weight of 8,000
⁶calcined kaolin clay available from KaMin (Macon, GA)
⁷processing aid available from Akrochem Corporation (Akron, OH)
⁸polymerized 2,2,4-trimethyl-1,2-dihydroquinoline available from Akrochem Corporation
⁹nitrosamine free vulcanizing agent available from Arkema (Colombes, France)
¹⁰vulcanizing accelerator available from Safic-Alcan (La Defense, France)
¹¹accelerator available from Akrochem Corporation

The prepared rubber stoppers were tested for their breakloose and gliding forces at 380 mm/min in borosilicate syringe glass barrels as per ISO 11040. The results are shown in Table 2.

TABLE 2

| | | | Time | | | |
|---|---|---|---|---|---|---|
| | | | After | | Mean | Mean |
| | | | Assembly | | Breakloose | Gliding |
| Example | Lamination? | Condition¹² | (Days) | N¹³ | (N)¹⁴ | (N)¹⁴ |
| 1 | Non- | Wash; RT | 2 | 8 | 22.6 | 17.8 |
| | laminated | Drying: | | | | |
| 2 | ETFE | 115° C. | | 9 | 24.8 | 18.6 |
| | Laminated | | | | | |
| 3 | Non- | | 7 | 9 | 34.4 | 22.4 |
| | laminated | | | | | |
| 4 | ETFE | | | 8 | 34.1 | 21.2 |
| | Laminated | | | | | |
| 5 | ETFE | Wash; RT | 2 | 15 | 41.5 | 23.4 |
| | Laminated | Drying: | | | | |
| | | 115° C. + | | | | |
| | | Wash: | | | | |
| | | 30 min- | | | | |
| | | 80° C. | | | | |

¹²Wash treatment conditions of the stoppers after molding to remove any trace of mold release from the molding operation.
¹³Number of stoppers tested for breakloose and gliding forces.
¹⁴Determined when tested in a 1 ml long glass syringe with dimensions and test methodology as per ISO 11040.

### Examples 6-8

### Testing of Sample Stoppers

Further properties of at least one of the ETFE partially laminated stopper (Example 6) prepared as described in Examples 1-5 and the non-laminated stopper (Example 7) prepared as described in Examples 1-5 were compared to the conventional butyl rubber stopper (Example 8) described above.

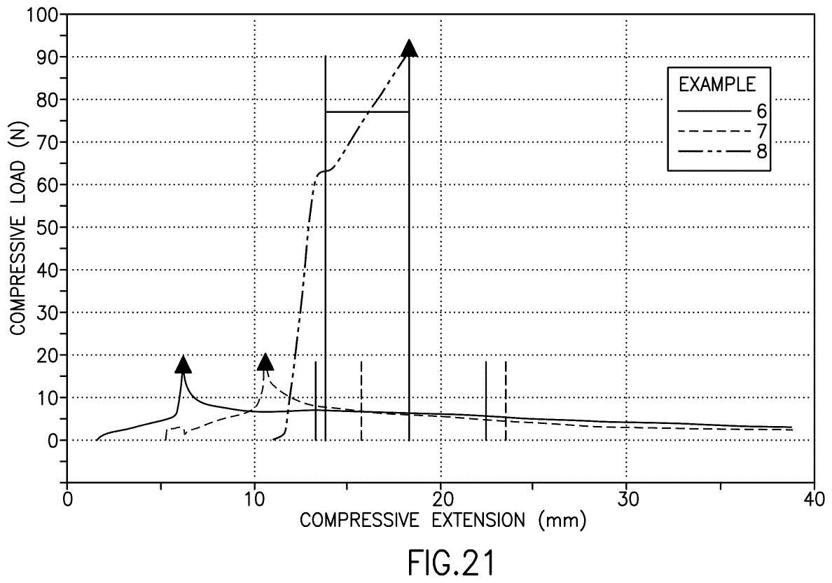

The stoppers of Examples 6-8 were tested for activation and gliding forces (AGF), and the graph of FIG. 21 shows the results of AGF profiles. Since the gliding forces continued to increase for the stopper of Comparative Example 8, the testing was stopped to avoid higher forces and potential shattering of the glass flange of the test fixture. The gliding forces for Examples 6 and 7, in contrast, did not continue to increase.

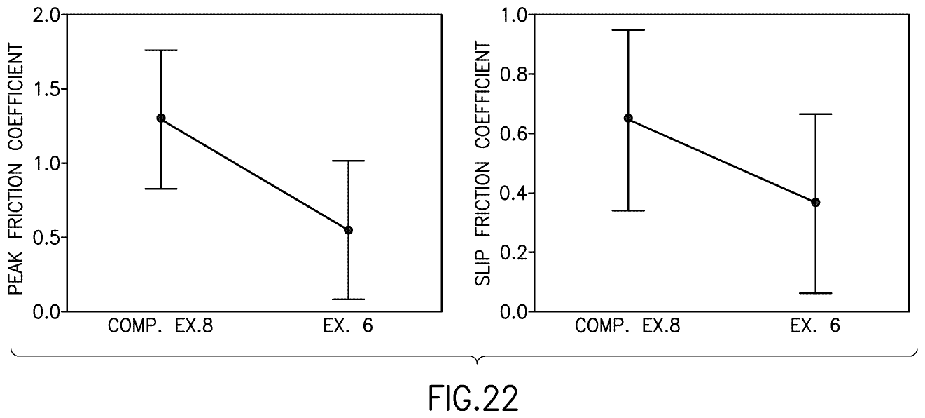

Static and dynamic friction coefficients were also measured for Example 6 and Comparative Example 8 against float glass using a ring and plate geometry on an Ares G2 rheometer at a constant normal force, which results are shown in FIG. 22. The stopper of Example 6 showed a meaningfully lower peak and slip friction coefficients compared to Comparative Example 8.

Leakage tests were performed according to ISO 11040 using 3 bars of pressure for 30 seconds on stoppers according to Examples 6-8. The stoppers of Examples 6-7 prepared using the rubber stopper described herein performed similarly well to the stopper of Comparative Example 8 (the conventional butyl rubber) by exhibiting no leakage failures in 42 samples tested (14 for stoppers of Example 6 and 28 for stoppers of Example 7).

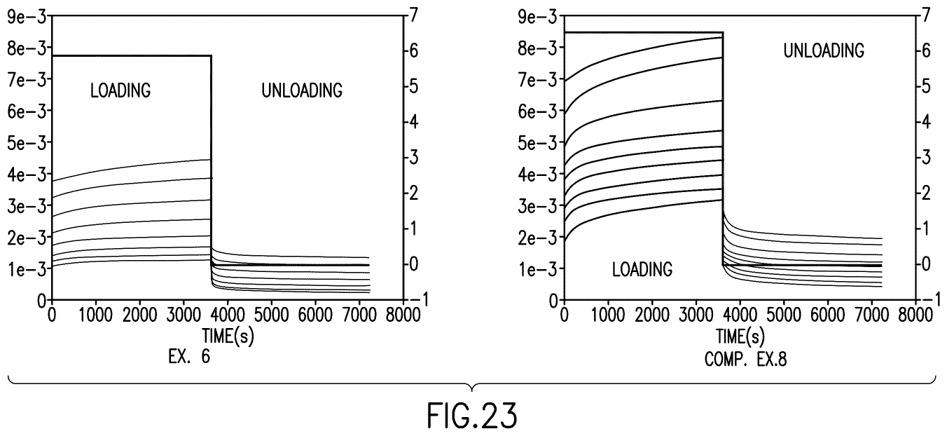

Example 6 and Comparative Example 8 were also tested for Dynamic Mechanical Analysis Time-Temperature Superposition (DMA-TTS) strain relaxation with a loading stress of 0.35 MPa with loading and unloading time of 3600 s over the range of 2° C. to 70° C. The results are shown in FIG. 23, with the left graph showing the strain relaxation of the stoppers of Example 6, and the right graph showing the strain relaxation of the stoppers of Comparative Example 8. As can be seen from FIG. 23, the stopper of Example 6 showed less strain relaxation under loading and unloading compared to Comparative Example 8.

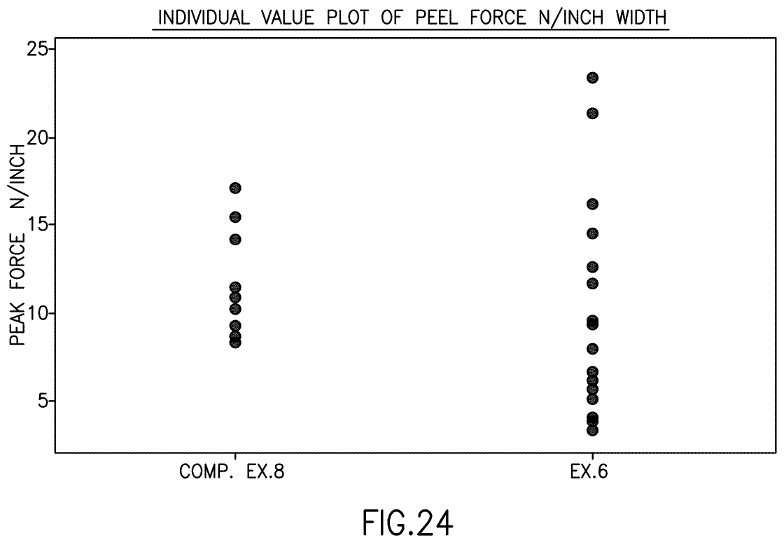

Example 6 and Comparative Example 8 were also peel-off tested based on ASTM D903 to test the bond strength between the rubber and the ETFE film laminated thereon. Samples were tested on an Instron tensile test bench at 10 mm/minute and 180° angle peel-off direction. FIG. 24 shows the results of the peel-off test. Both rubbers showed similar bond strength to the ETFE film.

Elements of one disclosed aspect can be combined with elements of one or more other disclosed aspects to form different combinations, all of which are considered to be within the scope of the present invention.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A medical device comprising a container defined by walls and having a first end having an opening, wherein the opening is sealed by a rubber component,
wherein the rubber component is formed from a self-lubricating rubber formed from a rubber composition comprising a halogenated isobutylene-isoprene co-polymer and at least one of (1) clay minerals or silica and (2) a liquid polyisoprene and butadiene homopolymer.

2. The medical device of claim 1 selected from the group consisting of a syringe assembly, drug cartridge, needleless injector, liquid dispensing device, liquid metering device, sample collection tube or plate assembly, catheter, and vial.

3. The medical device of claim 1, wherein the rubber component comprises a pierceable septum.

4. The medical device of claim 1, wherein the medical device comprises a drug delivery system for injecting a medicament, wherein the container comprises a syringe barrel configured to receive the medicament, wherein the rubber component comprises a stopper configured to slide against the walls within the syringe barrel from a pre-use position to a post-use position.

5. The medical device of claim 1, wherein the halogenated isobutylene-isoprene co-polymer comprises a chloro-isobutylene-isoprene co-polymer.

6. The medical device of claim 1, wherein the clay minerals comprise calcined magnesium silicate clay or aluminum silicate clay.

7. The medical device of claim 1, wherein the liquid polyisoprene and butadiene homopolymer has a weight average molecular weight of from 20,000 to 50,000 g/mol.

8. The medical device of claim 1, wherein the rubber composition comprises:
from 50 to 100 phr of the halogenated isobutylene-isoprene co-polymer;
from 30 to 60 phr of the clay minerals or silica; and
from 3 to 30 phr of the liquid polyisoprene and butadiene homopolymer.

9. The medical device of claim 1, wherein the walls comprise glass, stainless steel, or a polymeric material.

10. The medical device of claim 9, wherein the polymeric material comprises polypropylene and/or a cyclic polyolefin.

11. The medical device of claim 4, wherein the stopper and/or the walls are substantially free of a lubricant.

12. The medical device of claim 1, wherein the lubricant comprises a silicone oil.

13. The medical device of claim 1, wherein the halogenated isobutylene-isoprene co-polymer is non-staining.

14. The medical device of claim 1, wherein the rubber composition is cured to form the self-lubricating rubber by sulfur vulcanization.

15. The medical device of claim 1, wherein at least a portion of the rubber component is laminated by a polymer comprising ethylene tetrafluoroethylene (ETFE) and/or polytetrafluoroethylene (PTFE).
